(19) United States
(12) Patent Application Publication
(10) Pub. No.: US 2016/0242778 A1
(43) Pub. Date: Aug. 25, 2016

(54) CLAMP DEVICE AND ITS CLAMP UNIT

(71) Applicant: ANREI MEDICAL (HZ) CO., LTD., Hangzhou, Zhejiang (CN)

(72) Inventors: JIFEN XU, Hangzhou (CN); JOSHUA GLENN TRANTER, HANGZHOU (CN); LIZ AIPING SHEN, Hangzhou (CN); WEIHUA LI, Hangzhou (CN); CRAIG BRUCE BERKY, HANGZHOU (CN)

(21) Appl. No.: 14/382,419

(22) PCT Filed: Oct. 15, 2013
(86) PCT No.: PCT/CN2013/085211

§ 371 Date: Sep. 2, 2014

(30) Foreign Application Priority Data
Jun. 29, 2013 (CN) 201310272522.2

(51) Int. Cl. A61B 17/08; A61B 17/122; A61B 17/128; A61B 17/10

(52) U.S. Cl. CPC A61B 17/08; A61B 17/10; A61B 17/122; A61B 17/1285; A61B 2017/12004

(57) ABSTRACT

A clamp device and its clamp unit has a clamp unit; a end of the connecting piece is connected with the connecting tube and a front is connected movably to the clip holder, the connecting piece has a frangible portion; the unlocking piece is on the connecting piece and is stop-limited between the connecting piece and the connecting pipe; two clips are connected with the clip holder by a connecting pin; the clip holder is in the clamp tube and can move, the clip stop pin is fixed on the front of the clamp tube to limit the clip holder sliding out from the clamp tube; the clamp tube's wall has spring reeds, which is bent inward and cooperates with the locking surface of the clip holder to form a one-way locking structure; the unlocked piece is provided with a pushing pulling surface and a wedge surface to unlock the spring reed.

### FIELD OF THE INVENTION

The present invention relates to a clamp device used in endoscopic view, in particular, it relates a clamp unit of a clamp device for the endoscope view for wound closure, label and anchor.

### BACKGROUND OF THE INVENTION

With the development of endoscopic therapy technology, many gastrointestinal disorders, such as defects in the digestive tract mucosa, bleeding ulcers, bleeding arterioles, small polyps in the colon diverticulitis treatment, require using clamp device directly for treatment; the clamp device can also be instead the endoscope to be used with markers under the monitor by X-ray. The clamp device can also serve as anchoring device for feeding tubes and other equipment; it can also serve as closing processing device in a conservative treatment for the gastrointestinal perforation.

In current technology, the using of clamp device is to grip tissue to close wounds, to stop bleeding, to make marks, to anchor.

In the China patent application No. CN200980123274.7 the clip proximal end integrally fixed to the capsule, the end of the clamp jaws in the tube is fixed, thereby the clamp jaws cannot tilt, and thus the fixed end is easily bent to impact the open distance of the clip mouth. The connection between the clamp jaws and the push portion by spring reed, so that the clamp jaws cannot rotate relative to the push portion. This device do not have unlock features, pre-closure of the clamp jaws needs to maintain a pulling force, the clamp jaws cannot be unlock after the locking misuse.

In China patent application No. CN201220250652.7, this device does not have unlocking function, Pre-closing the clip is needed to be maintained by the suitable back tension, and the locking clip of misuse cannot be unlocked. Further, the elastic ring for holding the jaw closed, the manufacturing and assembly for this part are complicated; this device has no rotation mechanism.

In application No. CN201220160369.5 the clamp jaws and four-linking rod structure are held in the holder, the holder seat is pulled into the connector sleeve, the holder seat has no locking mechanism, when human tissue peristalsis, the clamp jaws is easy to slide out from the holder, eventually leads the clamp failure. Also, no rotation mechanism for this instrument.

### SUMMARY OF THE INVENTION

To solve the above technical problems, the present invention is to provide a reasonable structure, compact and reliable clamp device and clamp unit having an automatic pre-locking, unlocking ability, repositionable functions, etc.

To achieve the above objects, the present invention adopts the following technical solutions:

A clamp unit of a clamp device comprises a clip, a clip holder, a clamp tube, a clip stop pin, a connecting piece, a connecting pipe, an unlocking piece and connecting pin. Insert an end of the connecting piece into the connecting pipe and fix it therein, a front of the connecting piece is connected movably to the clip holder, and thereby the connecting piece is able to pull the clip holder moving backward. The connecting piece has a frangible portion, the unlocking piece is sleeved in the clam tube and is stop-limited between the connecting piece and the connecting pipe. Clip ends of the two clips are inserted into the clip holder and are connected therein through the clip connecting pin, then the clip holder is placed inside of the clamp tube and can move back and forth. The clip stop pin is fixed on the front of the clamp tube to limit the clip holder sliding out from the clamp tube. The clamp tube's wall has spring reeds, the spring reed is bent inward, which cooperates with the locking surface of the clip holder to form a one-way locking structure; the unlocked piece is provided with a pushing pulling surface to push the clip holder moving forward and with a wedge surface to unlock the spring reed.

For convenience of description, in the present specification the direction of the two clips extending axially from tube and open is as front, reverse direction is as back. Similarly, the parts closing to the clip are considered as front, on the contrary are considered as back. The clip stop pin is the stopper rod, bar, block, or any other shaped piece, which is able to limit the clip holder sliding out from clamp tube, it may be a separate component or may be formed directly on the clamp tube. The connecting pin of the present invention is the stopper rod, bar, block, or any other shaped piece which is able to position or limit the clip end of the clip, it may be a separate component, or can be directly fixed to the clip holder.

In this scheme, the clip holder is placed inside of the clamp tube and can relative axial move. The wall of the clamp tube has a resilient reed spring which cooperates with the locking surface of the clip holder to form a one-way locking structure. Whether it is pre-locking or final positioning clamp locking, the locking of clip holder needs not any outside force to maintain the locking state. The pre-locking structure is reliable and convenient. The unlocking piece may move relatively to the clip holder. When the clip holder is in the locking position, the unlocking mechanism takes no effect, but after the wedge portion of the unlocking piece is moved under the reed spring of the clamp tube by pushing, the reed spring is opened to let the clip holder moving forward to achieve unlocking.

Advantageously, the front end of the unlocking piece is connected with the clip holder in no turning possibility. The rotor of the operation unit drives the spring core shaft turning. The spring core shaft is connected with the connecting tube, the connecting pipe brings the unlocking piece turning, the unlocking piece brings the clip holder turning, and the clip holder brings the clips turning. By this turning operation of the clips a best grip angle is provide for improving the accuracy of the clamping operation.

Advantageously, the clip comprises a clip end, a connecting portion and a holding portion. The clip end has a slot, a connecting pin and the slot are in sliding fit, thus the connecting pin can move back and forth in the slot. The connection portion can be accommodated into the clamp tube. The clip holder has a wedge groove, the clip end of the clip can be moved vertically along the surface of the wedge groove. Thus, the clip holder uses a movable connection with the clip, both can relatively move forward and backward, up and down in a small range. When the clips are closed and tightened into the clamp tube, the two clip ends can move outwardly for reducing restriction to avoid yield; when the clips go out the clamp tube to open, the clip ends of two the clips can move inwardly, the angle between the two side clips is increased, thereby increase the two clips opening distance.

Advantageously, the unlocking piece is provided the wedge portion, which is movably inserted in the mounting groove of the clip holder. The wedge portion has a wedge surface, when the reed spring and the locking surface of the clip holder are in one way locking fit the wedging portion of the unlocking piece pushes the reed spring to open, then the clip holder can move forward. The wedge portion of the unlocking piece cooperates with the clip holder are in a circumferentially fixing fit and they can be moved axially to make an implementation of unlock or rotation function in simple and reasonable way.

A clamp device includes an operation unit, a clamp push unit and a clamp unite. The spring core shaft of the operating unit is connected with the connecting pipe of the clamp unit, the spring core shaft has spring tube outside which is connected with the operation unit. Other end of the spring core shaft is connected with the clamp tube holder, the clamp tube holder and the clamp tube are detachable connection.

Advantageously, the operation unit includes a handle, a slide handle, and a rotor. The slide handle can slide on the handle. The sliding handle is connected with the spring core shaft. The rotor is on the spring core shaft and is able to drive the spring core shaft to rotate, the spring core shaft can be moved in a hole in the rotor, and the spring tube of the clamp push unit is connected with the handle.

Advantageously, the push unit includes a clamp tube holder, a push open tube and a spring tube. The front end of the clamp tube holder has an elastic structure. The push open tube inserts into the clamp tube holder's inner hole thus the head of the clamp tube holder is enlarged. The enlarged head is connected with the connecting ring of the clamp tube movably. In natural state the outer diameter of the clip holder is smaller than the inner diameter of the connecting portion of the clamp tube; the push open tube is on the connecting tube, the connecting pipe is a tubular part with a flange, the diameter of the flange is greater than the inner diameter of the push open tube. Thus the fixing and releasing of the clamp tube are more convenient and reliable.

Since the present invention uses the above-mentioned technical solutions, the clip holder has automatic locking function. The pulling maintenance required by locking is avoided when pre-locking, while the analog effect of clamp portion is released, which facilitates medical judgment. The clip holder having unlocking feature provides unlocking function for wrong operation. The technical solution is simple and reasonable, stable and reliable. The clips can be repeated opening and closing, and the closed clamps can be repositioned many times.

### BRIEF DESCRIPTION OF THE DRAWING

FIG. 1 a cross section view of the structure of the present invention;

FIG. 2 is a perspective view of the clamp unit (eliminating clamp tube and clip stop pin);

FIG. 3 is a cross section view of the clamp portion (eliminating clamp tube and clip stop pin);

FIG. 4 is a perspective view of the clip;

FIG. 5 is a perspective view of the clip holder;

FIG. 6 is a perspective view of the clamp tube;

FIG. 7 is a perspective view of the connecting piece;

FIG. 8 is a perspective view of the connecting pipe;

FIG. 9 is a perspective view of the unlocking piece;

FIG. 10 is a cross section view of the clamp tube holder;

FIG. 11 is a cross sectional view showing the clip holder in pre-locking;

FIG. 12 is a perspective view showing the clip holder in pre-locking;

FIG. 13 is a cross-sectional view showing a clip holder in unlock;

FIG. 14 is a cross-sectional view showing a state of connecting piece is pulled down, the push open tube is pulled back;

FIG. 15 is a cross-section view of the clamp released state.

### DETAIL DESCRIPTION OF THE INVENTION

Below with reference to the specific embodiments of the invention have a detailed description.

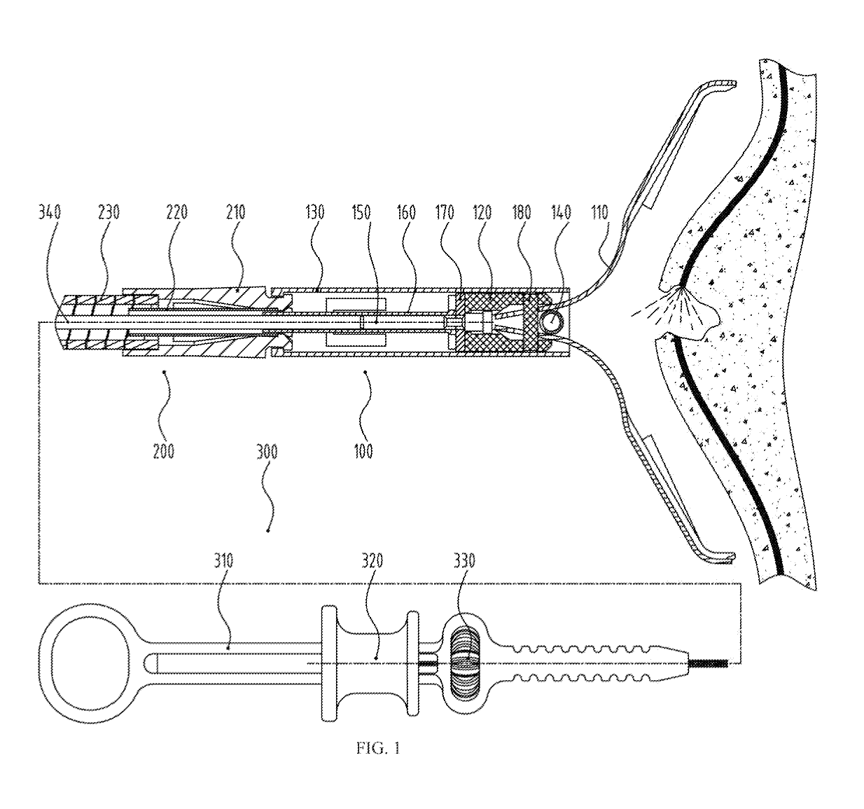

A clamp device shown in FIG. 1, which includes the operation unit 300, the clamp unit 100 and the clamp push unit 200. The clamp unit 100 is for holding the body tissue, the clamp push unit 200 is for pushing the clamp unit 100 into the body and connecting the clamp unit with the operation unit 300 for operating. One end of the spring core shaft 340 in the operation unit 300 is fixed connected with the connecting tube 160 of the clamp unit, the spring tube 230 of the push clamp unit 200 is on the sprig core shaft 340, one end of the spring tube 230 is connected with the operation unit and other end is connected with the clamp tube holder 210 which is disassembly connected with the clamp tube 130 of the clamp unit 100.

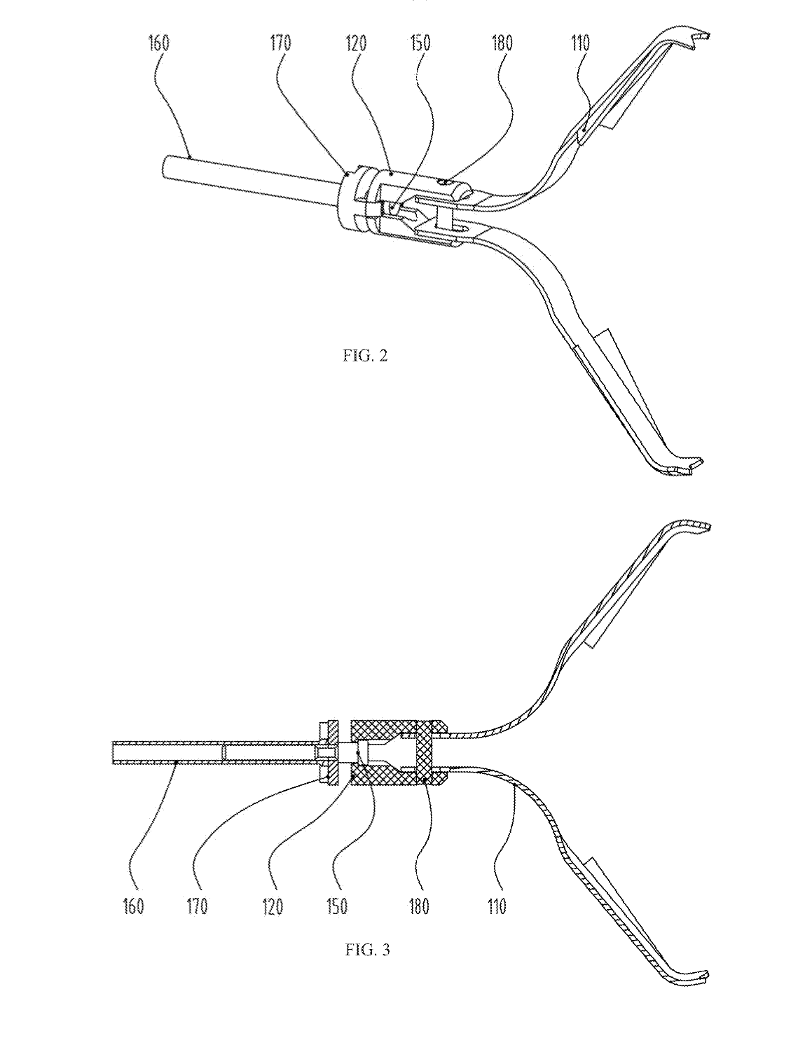

A clamp unit 100 of the clamp device as shown in FIG. 2, includes the clip 110, clip holder 120, clamp tube 130, clip stop pin 140, connecting piece 150, connecting tube 160, unlocking piece 170 and connecting pin 180. In the FIG. 2 and FIG. 3, the rear end of the connecting piece 150 is inserted into the connecting tube 160 and fixed therein, the front end of the connecting piece 150 is connected movably with the clip holder 120, and thereby the clip holder 120 can be pulled to move rearward. The connecting piece 150 has a frangible portion 151, the unlocking piece 170 is on the connecting piece 150 and is defined between an end of the connecting piece 150 and an end of the connecting tube 160. The clips 110 are connected with the clip holder by two clip ends 111 inserted in the holder 120 and the connecting pin 180. The clip holder 120 is placed inside the clamp tube 130 and can be moved back and forth, the clip stop pin 140 is fixed to the front portion of the clamp tube 130 to limit the clip holder to slide out of the clamp tube 130. The clamp tube 130 has a reed spring 132 which is inward and works with the locking surface 121 of the clip holder 120 to form a one-way lock structure. The unlocking piece 170 has a pushing and pulling surface 173 which can push the clip holder 120 forward movement and a wedge surface 171 which can push to open the reed spring 132 to achieve unlocking. The front end of the unlocking piece 170 can be connected with the clip holder 120 in a no turn-able way, the rear end of the unlocking piece 170 can be connected with the connecting tube 160 in turn-able way.

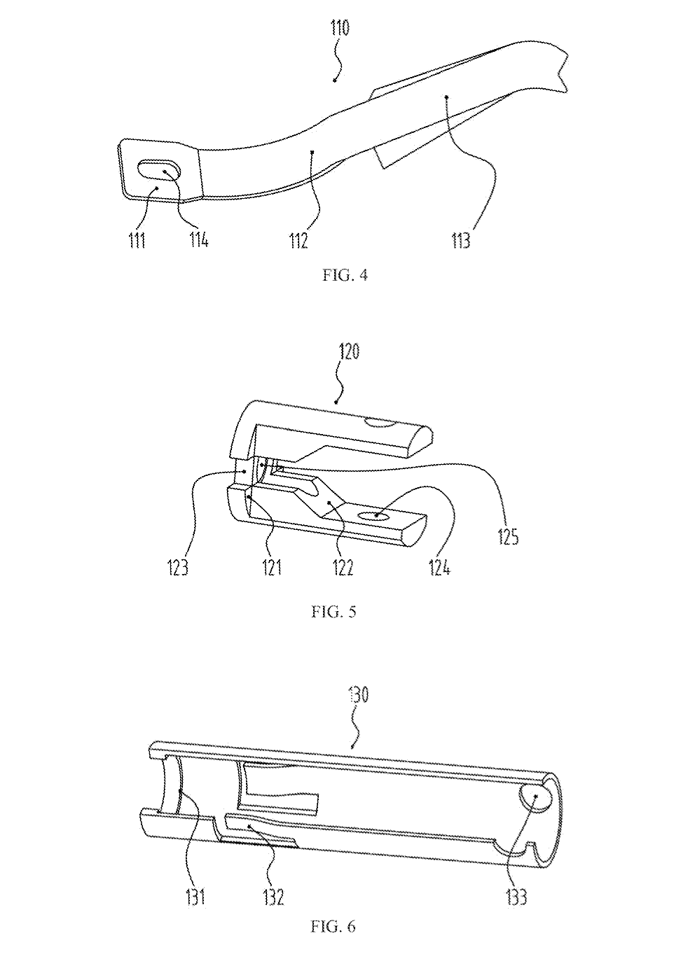

Shown in FIG. 4, the clip 110 includes a clip end 111, a connecting portion 112 and the holding portion 113. The clip end 111 is used for fixing the clip 112 on the clip holder 120. The holding portion is used for gripping tissue, the connection section 112 is used to connect the holding portion 113 with the clip end 110 and provides a holding span when the clamp portion 110 is opened. The end of clip end 110 has a slot 114, the fit tolerance between the connecting pin 180 and the slot 114 is movable fit, so that both one may do relative movement, the connecting portion 112 can be accommodated into the clamp tube 130.

Shown in FIG. 5, the clip holder 120 includes the locking surface 121, wedge groove 122, mounting groove 123, through holes 124 and connecting sink bore 125. The locking surface 121 cooperates with the reed spring 132 on the wall of the clamp tube 132 to form a push forward fixing in one direction. The wedge grooves 122 pushes the clip 110 forward to make the clip ends 110 to be closed along the surfaces of the wedge groove 112, thereby the two clips 110 generate an additional opening angle.

The connecting pin 180 used for connecting the clip 110 with the connecting pin 180 has an outer diameter which is smaller than the width of the slot 114. The connecting pin 180 is fixed in the through holes 124 of the clip holder 120.

As shown in FIG. 6, the clamp tube 130 includes the connection ring 131, reed spring 132, pin fixing hole 133. When the head 211 of the clamp tube holder 210 being supported to enlarge, the inner diameter of the connecting ring 131 is smaller than the diameter of the enlarged head 211 and larger than the diameter of the neck 212; when the head 211 of the clamp tube holder returning nature status the inner diameter of the connecting ring 131 is larger than the diameter of the head 211. The reed spring 132 has elasticity, the pin fixing hole 133 is for the clip stop pin 140. The clip stop pin 140 is a hollow tube mounted in the pin fixing hole 133, and then fixed with the clamp tube 130 through enlarging the hollow end of the clip stop pin 140.

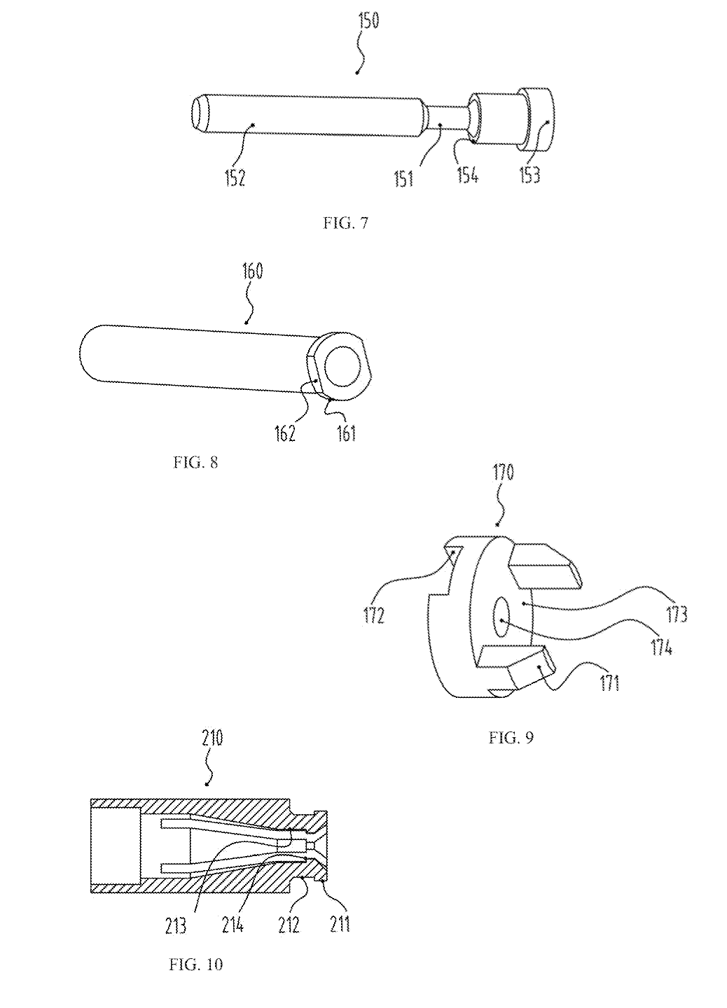

As shown in FIG. 7, the connecting piece 150 is a step shaft comprising a frangible portion 151, a rear end 152, a flange 153, and a shaft shoulder surface 154. The frangible portion 151 is set to be broken under certain pull force. The rear end 152 fixedly connected to the connecting tube 160 for push-pull transmission. The flange 153 is set in the connecting sink bore 125 of the clip holder 120. The shoulder surface 154 of the connecting piece 150 can pass through the clip holder 120 to contact with the end of the unlocking piece 170, mutually transfer force.

As Shown in FIG. 8, the connecting tube 160 is a tubular part with a flange 161 with two flat surfaces 162. The spring core shaft 340 is inserted into the inner hole of the connection tube 160 and fixed therein and also is fixedly connected with the connecting piece 150 to transfer push-pull and rotary torque. The diameter of flange 161 of the connecting tube 160 is greater than the inner diameter of the push open tube 220, the end surface of the flange 161 contacts with the bottom surface of the unlocking pieces, the plate surfaces 162 of the flange 161 enter the groove 172 of the unlocking piece 170, the torque transmission can be made.

As Shown in FIG. 9, the unlocking piece 170 includes the wedging portion 171, the groove 172, the pushing pulling surface 173, the through hole 174. The wedged portion 171 is mounted in the mounting groove 123 of the clip holder 120, the wedge portion has a wedge surface. When the reed spring 132 of the clamp tube 130 locking in one direction with the locking surface 121 of the clip holder 121, under pushing the wedge portion of the unlocking piece moves into the place under the reed spring 132 and pushes the reed spring 132 to open, thereby the stop for the clip holder 120 moving forward is released. The groove 172 of the unlocking piece 170 is used for setting the flange 161 of the connecting tube 160 and for transfer the push force and torque of the connecting tube. The pushing and pulling surface 173 connects with the connecting piece 150, clip holder 120 and transfers the pushing and pulling force. The inner diameter of the through hole 174 is larger than the diameter of the frangible portion 151 and rear end 152 and is smaller than the diameter of the shoulder surface 154.

The clamp push 200 unit includes a clamp tube holder 210, push open tube 220 and spring tube 230. The front end of the clamp tube holder 210 has a elastic folder structure, the push open tube 220 inserts into the inner hole 213 to enlarge the head 211, the enlarged head 211 passes the connecting ring 131 of the clamp tube 130 and is stopped therein. In natural state the outside diameter of the head 211 is less than the inner diameter of the connecting ring 131. The push open tube 220 is on the connecting tube 160, which has a flange 161, the outer diameter of the flange 161 is larger than the inner diameter of push open tube 220.

As Shown in FIG. 10, the clamp tube holder 210 includes a head 211, neck 212, internal hole 213, hole bottom 214. In the natural state the outer diameter of the head 211 is smaller than the inner diameter of the clamp tube 130, that is facilitate the separation of clamp tube. The clamp tube holder has resilient structure, the inner diameter of the internal hole 213 is less than the outer diameter of the push open tube 220. The hole bottom 214 is used for stopping the bottom surface of the push open tube 220. The inner diameter of the push open tube 220 is greater than the outer diameter of the connecting tube 160, is smaller than the outer diameter of the flange 161, when the connecting tube 160 being pulled to a specified position, the push open tube 220 can be pulled out. The spring tube 230 is connected with the clamp tube holder 210 and the handle 310. The inner diameter of the spring tube 230 is greater than the outer diameter of the spring core shaft 340 and the outer diameter of the connecting tube 160; the spring tube 230 has a spring core shaft 340. The spring tube 230 is used for pushing the clamp portion to the working position.

The operation unit 300 includes a handle 310, a slide handle 320, a rotor 330 and the spring core shaft 340. The slider 320 can slide on the handle 310, the slider 320 is connected to the spring core shaft 340, and the rotor 330 is on the spring core shaft and can turn the spring core shaft. The spring core shaft 340 can be moved in a hole of the rotor 330. The spring tube 230 is connected with the handle 310.

The handle 310 is connected to the spring tube 230, on the handle 310 the handle 320, rotor 330, spring core shaft 340 are set. The slide handle 320 is slidable on the rail of the handle 310 and connected with the spring core shaft 340, the slide handle can pull and push the spring core shaft. The rotor 330 can rotate on the handle 310 and is connected to the spring core shaft 340, the rotor 330 can drive the spring core shaft rotation. The spring core shaft may turn relative to the slide handle 320 and the handle 310, and may slide move forward or back relative to the rotor 330.

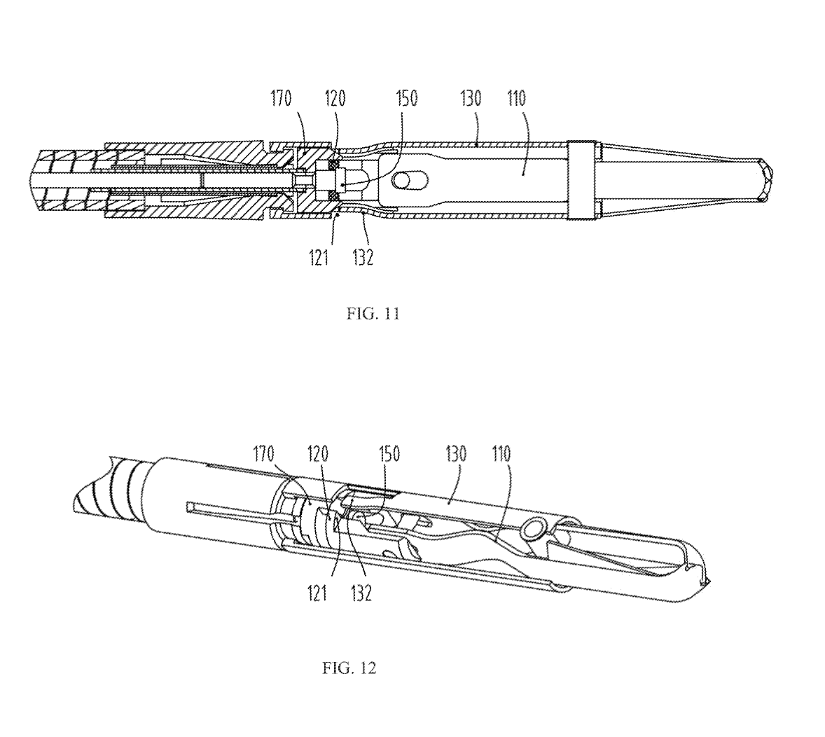

As shown in FIG. 11, FIG. 12, when the clip holder is in a pre-locked position, the connecting piece 150 pulls the clip holder 120 back, the clip 110 is withdrawn into the clamps tube 130, the reed springs 132 and the locking surface 121 is in a one-way lock, the unlock piece 170 relative to the clip holder 120 moves back to the place behind the reed spring 132.

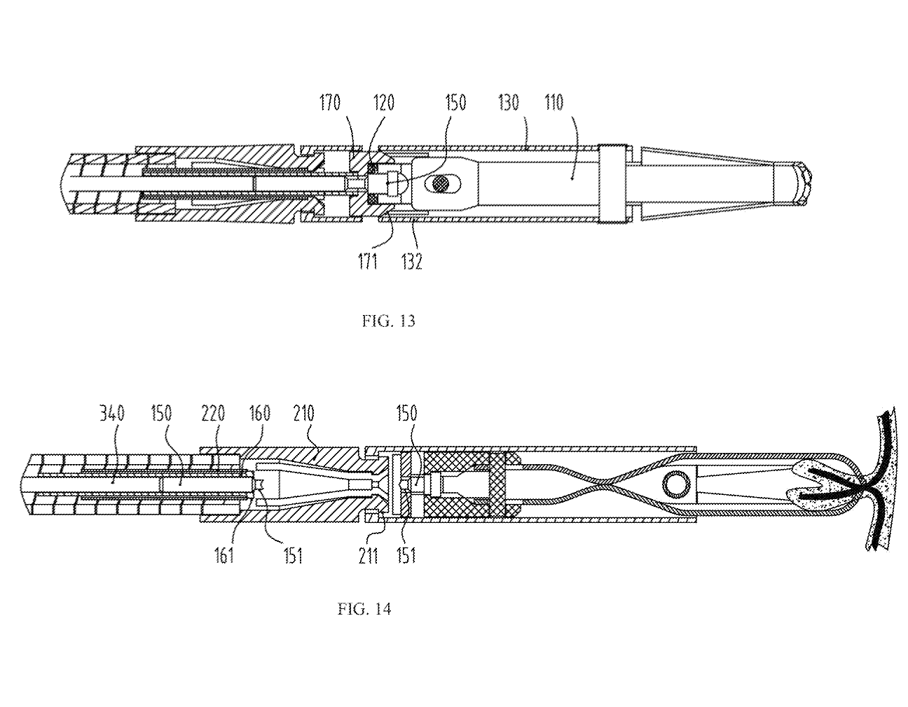

As shown in FIG. 13, the unlocking piece 170 is pushed forward to unlock, the wedge portion of unlocking piece 170 can wedges into the place under reed spring 132 and pushes the reed spring 132 to open, the clip hold 120 is unlocked and can be moved forward by the pushing of the unlocking piece, the clips 110 is reopened.

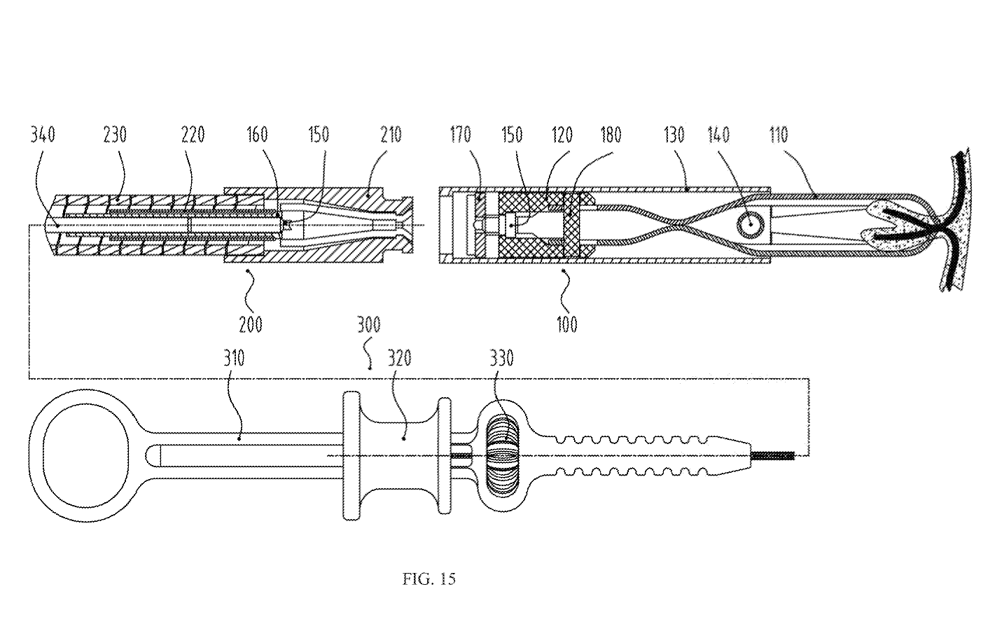

As Shown in FIG. 14, FIG. 15, the clamp unit is released, in the pre-locked state the spring core shaft 340 continues to pull the connecting piece 150 rearward, the clip holder 120 that is restricted as the clamp portion cannot be moved rearward, the frangible portion 151 of the connecting piece 150 is break by pulling and the push open tube 220 is pulled by the connecting tube to exit the inner hole 213, the head 211 of the clamp tube holder returns the nature state, so that the spring tube can bring the clamp tube holder 210 to leave the clamp tube, the clamp unit release is achieved.

What is claimed is:

1. A clamp unit used for a clamp device comprising:
a clip (110), a clip holder (120), a clamp tube (130), a clip stop pin (140), a connecting piece (150), a connecting tube (160), an unlocking piece (170) and a connecting pin (180);
a back end of the connecting piece (150) is inserted into the connecting pipe (160) and fixed therein, a front end of the connecting piece (150) is movably connected with the clip holder (120), thereby the clip holder (120) can be pulled backward, the connecting piece (150) has a frangible portion (151);
the unlocking piece (170) is on the connecting piece (150) and is defined between the connecting piece (150) and the connecting tube (160);
two clip ends (111) of the clamps 110 are inserted into the clip holder (120) and are connected with the clip holder (120) through the connecting pin (180), the clip holder(120)is placed in the clamp tube (130) and can move back and forth inside;
the clip stop pin (140) is fixed to a front portion of the clamp tube (130) to stop the clip holder 120 sliding out the clamp tube 130;
the clamp tube (130) has a reed spring (132), which is bent inwardly to cooperate with a locking surface 121 of the clip holder 120 to form a fitting structure of a one-way lock;
the unlocking piece (170) is provided a pushing pulling surface 173 for pushing the clip holder forward and a wedge portion 171 which can move forward to open the reed spring (132) to achieve unlock.

2. The clamp unit used for a clamp device according to claim 1, wherein the front end of the unlocking piece (170) is connected with the clip holder(120) in turn-able way, the back end of the unlocking pin (170) is connected with the connecting tube (160) in turn-able way.

3. The clamp unit used for a clamp device according to claim 1, wherein the clip (110) comprises a clip end (111), a connecting portion (112) and a holding portion (113), the clip end ((111) is provided with a slot (114), the connecting pin 180 is inserted into the slot 14 in movable fit tolerance, the connecting pins (180) is movable in the slot 114, the connecting portion (112) can be accommodated into the clamp tube (130).

4. The clamp unit used for a clamp device according to claim 1, wherein the clip holder (120) has a wedge groove (122), the clip end (111) can move along with the wedge grooves (122) vertically.

5. A clamp unit of a clamp device according to claim 1, wherein the unlocking piece (170) has a wedge portion 171, the wedge portion 171 is movable in a mounting grove (123) of the clip holder (120), the wedge portion 171 has a wedge surface, when the reed spring (132) of the clamp tube (130) cooperates with the locking surface (121) of the clip holder's (120) to become one way lock, the wedge portion 171 of the unlock piece (170) may be subjected to a forward thrust to push the wedge portion 171 to the reed spring (132)and to open the reed spring (132) to release the limit for the clip holder(120) moving forward.

6. A clamp device comprising:
a operating unit (300), a clamp push unit (200) and a clamp unit (100), a spring core shaft (340) is connected with a connecting tube (160) which is in a spring tube 230, one end of the spring tube 230 is connected with the operating unit 300, other end is connected with the clamp tube holder 210, which is detachably connected with the clamp tube 130.

7. The clamp device according to claim 6, wherein the operation unit (300) comprises a handle (310), a sliding handle (320), a rotor (330) and a spring core shaft (340), the slide handle (320) can slide on the handle (310), the slide handle (320) is connected with the spring core shaft (340), the rotor (330) is on the spring core shat (340) and is able to drive the spring core shaft (340) rotate, the spring core shaft (340) can move in a hole of the rotor (330), a spring tube of the clamp push unit is connected with the handle.

8. A clamp device according to claim 6, wherein the clamp push unit (200) includes a clamp tube holder (210), a push open tube (220) and a spring tube (230), the clamp tube holder (210) has a elastic folder structure, the push open tube 220 is inserted into a inner hole 213 of the clamp tube holder 210 in order to enlarge a head 211 of the clamp tube holder's (210), the enlarged end 211 is movably connected with a connecting ring 131 of the clamp tube 130, in natural state the diameter of the head 211 of the clamp tube holder is smaller than an inner diameter of the connecting ring 131, a push open tube is on the connecting tube 160 which has a flange 161, the diameter of the flange 161 is larger than an inner diameter of the push open tube 220.
